(19) United States
(12) Patent Application Publication
(10) Pub. No.: US 2009/0196807 A1
(43) Pub. Date: Aug. 6, 2009

(54) Sponge Sanitizer

(76) Inventor: Phillip E. Burns, Wilton Manors, FL (US)

Correspondence Address: GOLD & RIZVI, P.A., 600 N. PINE ISLAND ROAD, SUITE 450, PLANTATION, FL, 33324-1311, US

(21) Appl. No.: 12/024,259

(22) Filed: Feb. 1, 2008

(51) Int. Cl. A61L 2/18

(52) U.S. Cl. 422/300

(57) ABSTRACT

A sponge sanitizing apparatus includes a tray having a shallow basin and an integral receptacle. The basin is sized for retaining a defined quantity of liquid and for receiving a sponge therein. The integral receptacle defines a cavity for receiving a sterilant therein and also defines a void fluidically communicative with the basin and the cavity to allow the free flow of liquid between the cavity and the basin.

### BACKGROUND OF THE INVENTION

1. Field of the Invention

The present invention relates to sanitation in general and more particularly to an apparatus for sanitizing sponges such as sponges used in a household.

2. Discussion of the Related Art

Consideration for maintaining sanitary conditions in one's surroundings has increased significantly in today's society. To understand the emphasis placed on such consideration one only needs to view the various cleaning agents and products found on the shelves of retail establishments as well as the constant publicity about new and difficult to eradicate micro-organisms. The area to which a person is most exposed is the household, and when one thinks of household germs, the bathroom is the area that first comes to mind. However, kitchens consistently come up dirtier.

A significant number of people don't seem to worry about or even believe that the home kitchen is such a haven for dangerous microbes. During food preparation both raw meat and fresh vegetables are typically handled for a single meal. Without maintaining proper sanitary conditions there is virtual certainty that the foods will be cross-contaminated. Many individuals will make a concerted effort to keep the kitchen “clean” by wiping up at frequent intervals during the food preparation process, after every preparation session, and even at other times during the day. Researchers have discovered that people who had the cleanest-looking kitchens were often the dirtiest. Because these people were “cleaning” and wiping up so often, they were actually spreading bacteria over all the surfaces they tried to clean. Consequently, bacteria such as E. coli, salmonella, campylobacter, bacillus, listeria, staphylococci, and streptococci were being spread to a wide variety of surfaces such as countertops, faucets, sinks, refrigerator door handles, and cupboard doors and handles, virtually any exposed surface in the kitchen.

Unless sponges are replaced or disinfected regularly, they provide a breeding ground for microbes that make a home in their pores. These microbes use food residues for their growth. Within a week of use, the sponge can contain billions of bacteria. If these sponges are used for wiping surfaces, the sponge actually spreads bacteria. If the sponge is contaminated with pathogenic bacteria, spreading those bacteria makes it easier for others to pick up the contamination and possibly become ill.

Those most prone to contracting illnesses caused by those bacteria are the very young, the very old, and those with severely weakened immune systems. These bacterial pathogens are indeed a very serious problem. Just in the United States, the diseases they cause kill thousands of people each year with tens of thousands more debilitated for periods of time to fight off and recover from the illnesses. Further, the vast majorities of these illnesses occur or are contracted in the common household.

The number one culprit in spreading these microbes is the common cellulose sponge followed by the dishcloth. While dishcloths are typically laundered on a regular basis and are thus effectively sanitized, the cellulose sponge is typically used till worn out over a period of weeks or months without any attempt at sanitization. If the sponge is allowed to dry, a large portion of the bacteria will die, but a significant and measurable number of bacteria will even survive on a dry sponge. Because of its great absorbency, a sponge will stay wet or damp for a significant period of time, and if it is repeatedly used over regular periods, the sponge may never thoroughly dry. The harmful bacteria can survive up to two weeks on a damp sponge.

Therefore, a concerted effort must be put forth to continually sanitize the sponge. One method recognized as effective in sanitizing a sponge is to dampen the sponge and place it in a microwave oven for approximately one minute. While this may be effective, not every household owns a microwave, and even though the cost of ownership has dramatically lessened over the years, a microwave oven is still an expensive solution that not every household can afford. However, the most effective way to reduce the amount of bacteria on the sponge is to soak the sponge in bleach for a short period of time.

Thus what is desired is an apparatus that provides an easy and convenient means of introducing a bleach or bleach equivalent solution to a sponge for the purpose of sanitizing the sponge on a regular basis.

### SUMMARY OF THE INVENTION

The present invention is directed to a sponge sanitizing apparatus that satisfies the need for an effective way to maintain sponges in a sanitary manner. The sanitizing apparatus includes a tray having a shallow basin and an integral receptacle. The basin is sized for retaining a defined quantity of liquid and for receiving a sponge therein. The integral receptacle defines a cavity for receiving a sterilant therein and also defines a void fluidically communicative with the basin and the cavity to allow the free flow of liquid between the cavity and the basin.

Another aspect of the present invention is a sponge sanitizing apparatus including a tray defining a shallow basin sized for retaining a defined quantity of liquid and for receiving a sponge therein. The tray also forms an integral receptacle that further defines a cavity for receiving a sterilant therein and also defines a void fluidically communicative with the basin and the cavity to allow the free flow of liquid between the cavity and the basin. A sanitizing cartridge is received in the receptacle and defines at least one aperture through the cartridge and in registration with the void.

Yet another aspect of the present invention is a sponge sanitizing apparatus including a tray defining a shallow basin sized for retaining a defined quantity of liquid and for receiving a sponge therein. First and second receptacles are integrally formed in the tray, wherein each receptacle defines a cavity for receiving a sterilant therein, and each said receptacle further defines a void fluidically communicative with the basin and the cavity to allow the free flow of liquid between the cavity and the basin. A first sanitizing cartridge including a first sterilant is received in the first receptacle. The cartridge defining at least one aperture therethrough and in registration with the first void. A second sanitizing cartridge including a second sterilant of higher intensity than the first sterilant is received in the second receptacle and defines at least one aperture therethrough in registration with the second void.

These and other features, aspects, and advantages of the invention will be further understood and appreciated by those skilled in the art by reference to the following written specification, claims and appended drawings.

### BRIEF DESCRIPTION OF THE DRAWINGS

For a fuller understanding of the nature of the present invention, reference should be made to the accompanying drawings in which:

FIG. 1 is a perspective view of a sponge sanitizing apparatus embodying the present invention;

FIG. 2 is a cross-sectional view of the sponge sanitizing apparatus shown in FIG. 1 and taken along the line 2-2, FIG. 1;

FIG. 3 is an exploded partial perspective view of the sterilant receptacle of the sponge sanitizing apparatus further illustrating a removable cover insert;

FIG. 4 is a cross-sectional elevation view of the sterilant receptacle shown in FIG. 3 and taken along the line 4-4, FIG. 3;

FIG. 5 is an exploded perspective view of one configuration of a sanitizing cartridge containing a sterilant for being received in the receptacle of the sponge sanitizing apparatus;

FIG. 6 is a cut-away view of the cartridge of FIG. 5 illustrating the rotating ability of the cover with respect to the base to regulate the aperture size at the base of the cartridge;

FIG. 7 is an cross-sectional elevation view of the sanitizing cartridge of FIG. 5;

FIG. 8 is a perspective view of an alternate embodiment of the sponge sanitizing apparatus;

FIG. 9 is an exploded perspective view of a sponge sanitizing apparatus for attachment onto a separate tray;

FIG. 10 is a cross-sectional elevation view of the sponge sanitizing apparatus of FIG. 9 taken along the Line 10-10, FIG. 9.

FIG. 11 is a perspective view of a molded sponge sanitizing apparatus for attachment onto a separate tray;

FIG. 12 is a cross-sectional elevation view of the receptacle portion of the sponge sanitizing apparatus of FIG. 11 taken along the Line 12-12, FIG. 11.

Like reference numerals refer to like parts throughout the several views of the drawings.

### DETAILED DESCRIPTION OF THE PREFERRED EMBODIMENT

For purposes of description herein, the terms “upper”, “lower”, “left”, “rear”, “right”, “front”, “vertical”, “horizontal”, and derivatives thereof shall relate to the invention as oriented in FIG. 1. However, one will understand that the invention may assume various alternative orientations and step sequences, except where expressly specified to the contrary. Therefore, the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

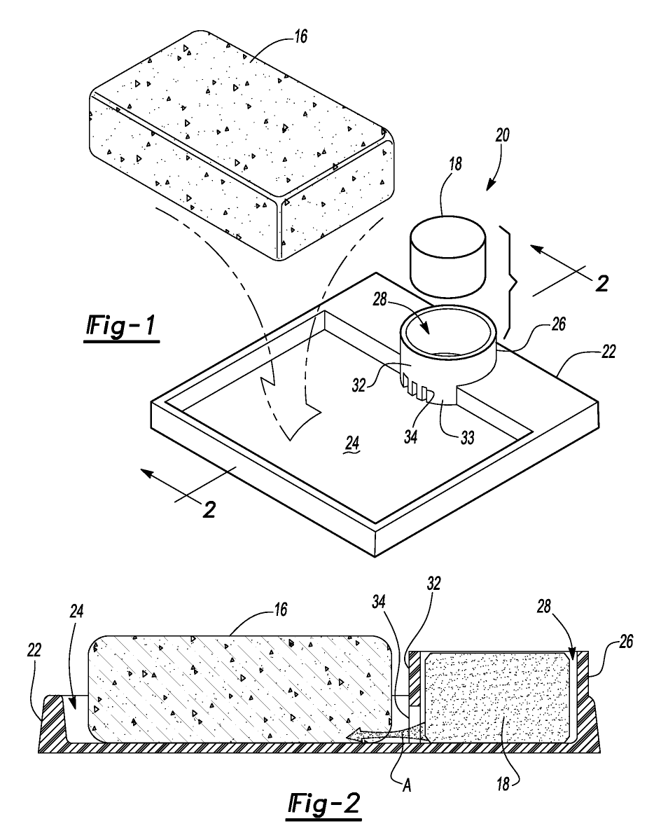

Turning to the drawings, FIGS. 1-2 show a sponge sanitizing apparatus 20 which is one of the preferred embodiments of the present invention and illustrates its various components. Apparatus 20 includes a molded tray 22 that defines a basin 24. Basin 24 can be of any shape but is sufficiently sized to receive a sponge 16 therein. Tray 22 also defines a receptacle 26 integrally formed therein. Receptacle 26, as illustrated in FIG. 1 has a cannular shape and defines a cavity 28 for receiving a sterilant 18, such as a chlorine bleach, here shown in tablet form. Receptacle 26 includes a wall 32 substantially separating basin 24 from cavity 28. A base 33 of wall 32 includes one or more more apertures 34 therethrough to permit fluidic communication between basin 24 and cavity 28. In use, sterilant 18 is placed in cavity 28 of receptacle 26 and a quantity of liquid such as water is put in basin 24. The liquid flows through apertures 34 to contact and dissolve sterilant tablet 18. The sterilant solution can then leach out (FIG. 2, arrow “A”) through apertures 34 into basin 24 where the sterilant solution is soaked into sponge 16 thus sanitizing sponge 16. Basin 24 has sufficient depth to contain a quantity of liquid to adequately soak into sponge 16 to perform the sanitizing function.

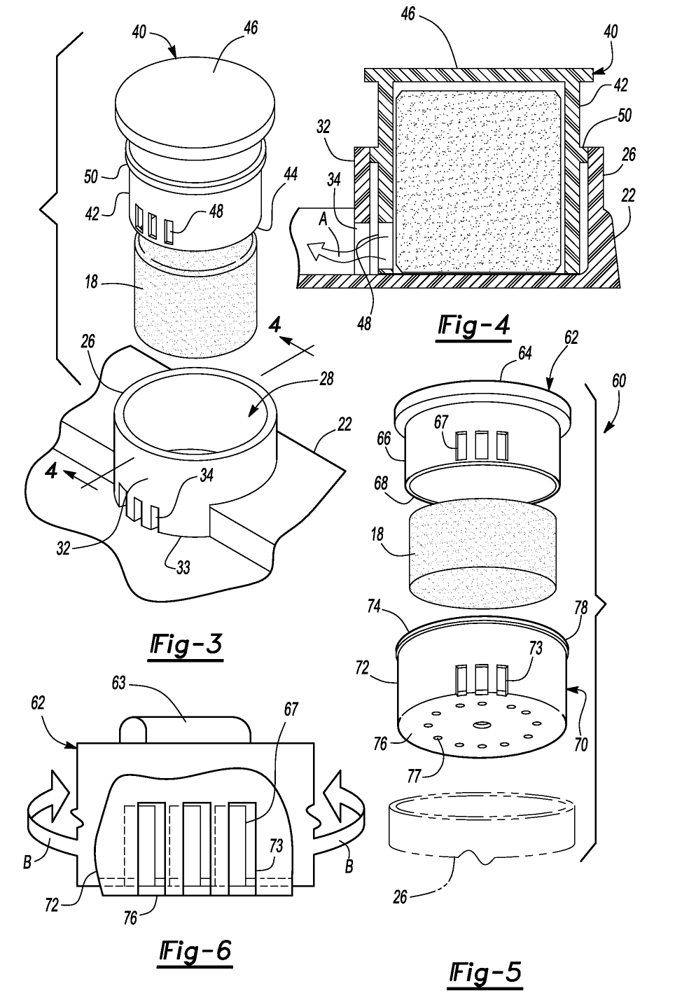

FIGS. 3-4 illustrate the inclusion of a cover insert 40 that is received in cavity 28 of receptacle 26. Cover insert 40 has a cannular body 42, an open bottom end 44, and an enclosed top end 46. Cover insert 40 functions to cover the sterilant 18 in cavity 28. Cannular body 42 generally extends to the bottom of receptacle 26 and has an inner diameter sufficient large to accommodate the physical configuration of sterilant 18. One or more apertures 48 are defined by cannular body 42 to correspond with apertures 34 at base 33 of receptacle 26. Cannular body 42 can also include a circumferential seal 50 about an exterior of cannular body 42. In the most preferred embodiment, circumferential seal 50 is integrally molded with cover insert 40 and has an outer diameter marginally smaller than the diameter of cavity 28 so that cover insert 40 can be closely received in receptacle 26. Circumferential seal 50 typically prevents the out-gassing of sterilant 18 from the top of receptacle 26. Typically, cover insert 40 is rotatable within receptacle 26 to move apertures 48 into and out of registration with apertures 34 thus regulating the flow (FIG. 4, Arrow “A”) of liquid sterilant from cavity 28 to basin 24 (FIG. 1).

Alternatively, cover insert 40 can be configured as an enclosed cartridge (not shown) wherein bottom 44 is enclosed with sterilant 18 encapsulated within at the time of manufacture. Such a cartridge, like cover insert 40, includes apertures 48 to permit the leaching of sterilant to basin 24 as previously described. Configuring cover insert 40 as a cartridge with sterilant 18 encapsulated therein has the advantage of easy replacement in receptacle 26 by the user. Such a cartridge also limits the user's direct contact with sterilant 18.

FIGS. 5-6 illustrate an alternate sanitizing cartridge 60. Cartridge 60 has a cover 62 separable from a base 70. Base 70 has a cannular body 72 with a closed bottom end 76 and an open upper end 74 for receiving sterilant 18 therein. Cannular body 72 defines one or more apertures 73 to permit the flow of fluid therethrough. Apertures 73 are configured to register with apertures 34 of receptacle 26 (FIG. 3). Closed bottom end 76 can optionally define a plurality of apertures 77 for additional fluid flow. Cannular body 72 can also include a circumferential seal 78 at or near open upper end 74 to be closely received in receptacle 26.

Cover 62 generally has a cannular body 66 with a closed upper end 64, an open bottom end 68, and cannular body 66 defining one or more apertures 67 configured to register with apertures 73 of base 70. Closed upper end 64 can also include a handle 63 for ease of handling by the user. Cannular body 66 of cover 62 is telescopically received in cannular body 72 of base 70 to enclose sterilant 18 therein. As illustrated in FIG. 6, cover 62 is rotatable within base 70 such that apertures 67 of cover 62 can be rotated into and out of registration with apertures 73 of base 70 (as indicated by arrows “B”).

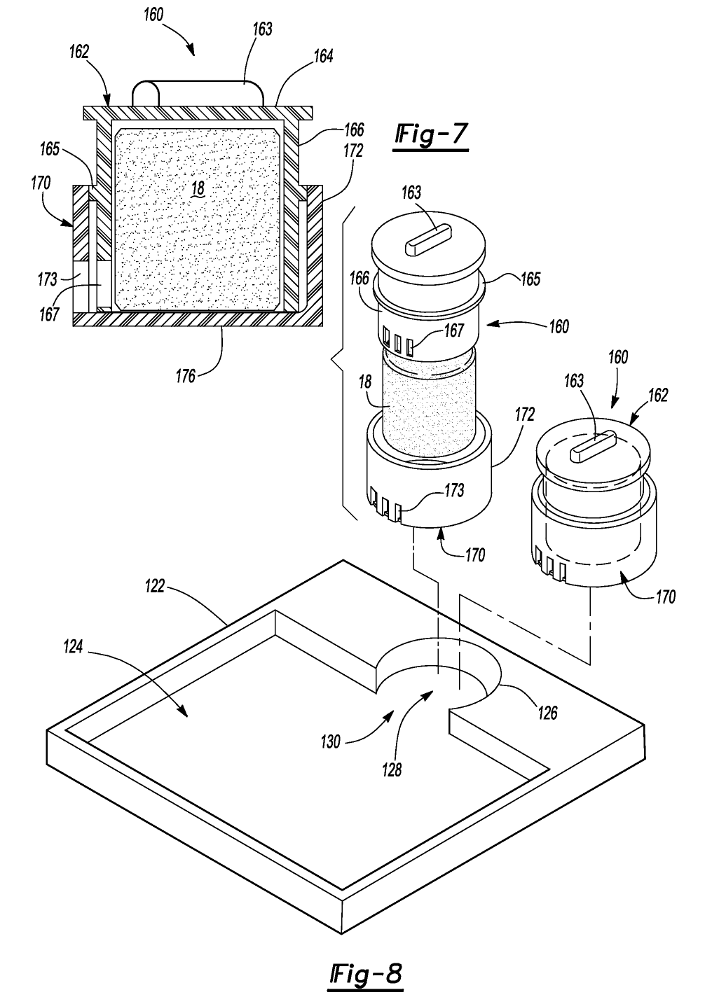

FIGS. 7-8 illustrate an alternate embodiment sponge sanitizing apparatus 120. Apparatus 120 includes a molded tray 122 that defines a basin 124 for receiving a sponge in the same manner as basin 24 receives sponge 16 in FIG. 1. Tray 120 also includes a molded receptacle 126 that defines a cavity 128 for receiving a sanitizing cartridge 160. A void 130 is defined between basin 124 and cavity 128 such that basin 124 and cavity 128 are in fluid communication one with the other. Void 130 may be such as to provide a small channel between basin 124 and cavity 128 or to allow a portion of cartridge 160 to protrude from cavity 128 into basin 124.

Cartridge 160 is similar in construction to cartridge 60 of FIG. 5. Cartridge 160 includes a separable cover 162 and base 170. Base 170 has a cannular body 172 with an enclosed bottom end 176 and defines one or more apertures 173 through cannular body 172 proximate to enclosed bottom end 176. A sterilant 18 is place within base 170 and cover 162 is telescopically received in cannular body 172. Cover 162 has a cannular body 166, an enclosed top end 164, and an open bottom end for telescoping over sterilant 18. Cover cannular body 166 defines one or more apertures 167 and is rotatable within base 170 such that apertures 167 can be rotated into and out of registration with apertures 173 in base 170. Cannular body 166 of cover 162 further includes a circumferential seal 165 therearound and is closely received in cannular body 172 of base 170 to inhibit the out-gassing of sterilant vapors from the dissolving sterilant tablet 18.

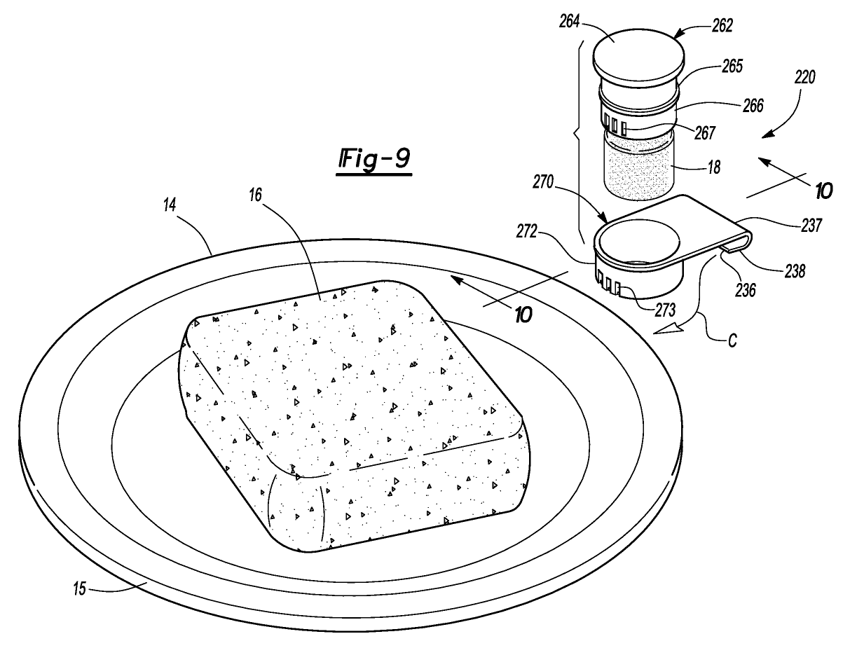

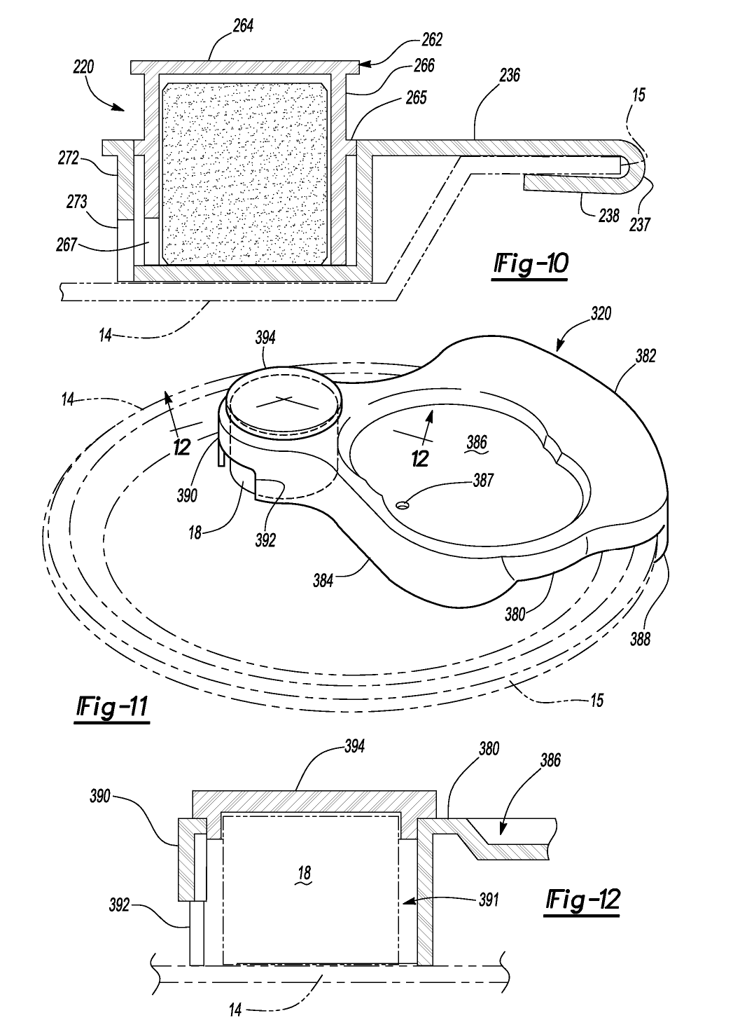

Turning now to FIGS. 9-10, a sponge sterilizing apparatus 220 that is attachable to a shallow tray, basin, plate or similar fluid retaining structure 14 is shown. Apparatus 220 has a base 270 and a cover 262. Base 270 is similar in construction to base 170 and cover 162 of FIGS. 7-8 wherein like features are similarly numbered. Base 270 has a cannular body 272 defining one or more apertures 273 proximate to an enclosed bottom. A sterilant 18 is received in cannular body 272, and cover 262 is telescopically received in base 270. Cover 262 is substantially identical to cover 162 of FIGS. 7-8 wherein its features are similarly numbered with a preceding numeral “2” instead of “1”.

Apparatus 220 has an attachment arm 236 that extends horizontally from an upper portion of base 270 and is positioned above apertures 273. Attachment arm 236 has at a distal end 237 an engagement hook 238. Engagement hook 238 receives a portion of edge 15 of fluid retaining structure 14 to secure apparatus 220 to structure 14 as indicated by arrow “C” in FIG. 9. When attached to fluid retaining structure 14, apertures 273 are proximate to the bottom of structure 14 to permit the flow of fluid in structure 14 to flow through apertures 273.

Yet another embodiment of a sponge sanitizing apparatus 320 attachable to a separate fluid retaining structure is illustrated in FIGS. 11-12. Apparatus 320 includes a molded tray or body 382 that has a receptacle 390 formed at a proximal end 384 and one or more engagement hooks 388 formed at a distal end 382. Engagement hooks 388 capture edge 15 of fluid retaining structure 14 for the purpose of securing apparatus 320 to structure 14. Body 380 defines a depression 386 that is sufficiently large to support either a bar of soap or a liquid soap dispenser (not shown) therein. A drain aperture 387 is defined in depression 386 to allow any liquid pooled in depression 386 to drain into structure 14. Receptacle 390 defines an integrally molded cavity 391 for receiving a sterilant 18 therein and further defines an aperture 392 to permit fluid communication between cavity 391 and structure 14. Receptacle 390 and body 380 are molded in such a manner that depression 386 and cavity 391 are fluidically isolated one from the other. A removable cover 394 is formed to be at least partially received in cavity 391 to cover the sterilant 18 within cavity 391. Alternatively a cover insert such as cover insert 40 as shown in FIG. 3 can be utilized in lieu of cover 394. Further, a cartridge such as cartridges 60 and 160 as illustrated in FIGS. 5 and 8 can also be used in conjunction with apparatus 320.

Those skilled in the art will readily recognize that the various covers and cartridges illustrated and described above can be used or adapted to the various fixed or removable apparatuses while adhering to the inventive concepts described herein. Further, any of the above described apparatuses can include a second receptacle in addition to the disclosed receptacle. One of the receptacles could include a mild sterilant and the other would contain a more intense sterilant such as bromine.

In use of apparatuses 20, 120, 220, or 320, a user will place a sterilant 18 in the respective receptacle (either directly or by way of one of cartridges 60 or 160). A volume of fluid such as water is introduced into the basin and the receptacle cover or cartridges are configured to permit fluid flow between the basin and the receptacle thus creating a sterilizing fluid. The volume of fluid should be such as to thoroughly soak the sponge to be sanitized. The sponge can remain in operating relationship with the apparatus or can be removed, wrung out, and allowed to dry after a set period to insure that the sponge has been sanitized. A new volume of fluid can be introduced for each subsequent sanitizing sequence. Once the sterilant 18 has been depleted, a new sterilant 18 or a new cartridge containing a sterilant 18 can be introduced to the apparatus.

The above description is considered that of the preferred embodiments only. Modifications of the invention will occur to those skilled in the art and to those who make or use the invention. Therefore, it is understood that the embodiments shown in the drawings and described above are merely for illustrative purposes and are not intended to limit the scope of the invention, which is defined by the following claims as interpreted according to the principles of patent law, including the doctrine of equivalents.

We/I claim:

1. A sponge sanitizing apparatus comprising:
a tray defining a shallow basin sized for retaining a defined quantity of liquid and for receiving a sponge therein; and
a receptacle integrally formed in said tray, said receptacle defining a cavity for receiving a sterilant therein, said receptacle further defining a void fluidically communicative with said basin and said cavity to allow the free flow of liquid between said cavity and said basin.

2. The sponge sanitizing apparatus of claim 1 wherein said sterilant is a solid tablet comprising in part a water soluble sterilant compound.

3. The sponge sanitizing apparatus of claim 1 wherein said receptacle includes a wall separating said cavity from said basin, said wall further defining at least one wall aperture at a base thereof, said aperture defining said void for fluidic communication.

4. The sponge sanitizing apparatus of claim 3 wherein said receptacle wall has a cannular shape.

5. The sponge sanitizing apparatus of claim 4 further including a cover insert received in said receptacle, said cover insert comprising a cannular body, said cannular body having an open bottom end for insertion into said cavity and an enclosed top end, said cannular body further defining at least one aperture therethrough, said at least one insert aperture sized and positioned to register with said aperture in said base of said receptacle wall.

6. The sponge sanitizing apparatus of claim 5 further including a circumferential seal extending about an exterior of said cannular body, said circumferential seal closely received in said cannularly shaped receptacle wall.

7. The sponge sanitizing apparatus of claim 6 wherein said circumferential seal is a rigid ring integrally formed with said cannular body.

8. The sponge sanitizing apparatus of claim 5 wherein said cover insert includes a handle affixed to said enclosed top end.

9. The sponge sanitizing apparatus of claim 5 wherein said cover insert is axially rotatable with respect to said receptacle, for selective rotation of said at least one insert aperture into and out of registration with said at least one receptacle wall aperture.

10. The sponge sanitizing apparatus of claim 5 wherein said tray includes:
a first receptacle in fluid communication with said basin and a first cover insert received therein for retaining a first sterilant; and
a second receptacle substantially identical with said first receptacle, said second receptacle in fluid communication with said basin and a second cover insert received therein for retaining a second sterilant.

11. The sponge sanitizing apparatus of claim 4 further including a sanitizing cartridge received in said receptacle, said sanitizing cartridge comprising:
a cartridge base for receiving therein a water soluble sterilant, said base having a cannular body, an open upper end, and a closed bottom, said cartridge base closely received in said receptacle and defining at a base thereof at least one aperture in said cannular body oriented in registration with said receptacle wall aperture; and
a cartridge cover telescopically received in said cartridge base and having a cannular body, a closed upper end, and an open bottom end, said cannular body defining at least one aperture therethrough sized and positioned to register with said at least one aperture in said cannular body of said cartridge base.

12. The sponge sanitizing apparatus of claim 11 wherein said cartridge cover is rotatable within said cartridge base.

13. The sponge sanitizing apparatus of claim 11 further including a circumferential seal extending about an exterior of said cannular body of said cartridge base, said circumferential seal closely received in said cannularly shaped wall of said receptacle.

14. The sponge sanitizing apparatus of claim 13 wherein said circumferential seal is a rigid ring integrally formed with said cannular body of said cartridge base.

15. The sponge sanitizing apparatus of claim 11 wherein said cartridge cover includes a handle affixed to said closed upper end.

16. The sponge sanitizing apparatus of claim 11 wherein said closed bottom of said cartridge base defines a plurality of apertures therethrough.

17. A sponge sanitizing apparatus comprising:
a tray defining a shallow basin sized for retaining a defined quantity of liquid and for receiving a sponge therein;
a receptacle integrally formed in said tray, said receptacle defining a cavity for receiving a sterilant therein, said receptacle further defining a void fluidically communicative with said basin and said cavity to allow the free flow of liquid between said cavity and said basin; and
a sanitizing cartridge received in said receptacle, said sanitizing cartridge defining at least one aperture therethrough and in registration with said void.

18. The sponge sanitizing apparatus of claim 17 wherein said sanitizing cartridge comprises:
a cartridge base for receiving therein a water soluble sterilant, said base having a cannular body, an open upper end, and a closed bottom, said cartridge base closely received in said receptacle and defining at a base thereof at least one aperture in said cannular body oriented in registration with said void; and
a cartridge cover telescopically received in said cartridge base and having a cannular body, a closed upper end, and an open bottom, said cannular body defining at least one aperture therethrough and sized and positioned to register with said at least one aperture in said cannular body of said cartridge base.

19. The sponge sanitizing apparatus of claim 18 wherein said cartridge cover is rotatable within said cartridge base.

20. The sponge sanitizing apparatus of claim 18 further including a circumferential seal extending about an exterior of said cannular body of said cartridge cover, said circumferential seal closely received in said cannular body of said cartridge base.

21. The sponge sanitizing apparatus of claim 20 wherein said circumferential seal is a rigid ring integrally formed with said cannular body of said cartridge cover.

22. The sponge sanitizing apparatus of claim 18 wherein said cartridge cover includes a handle affixed to said enclosed top end.

23. The sponge sanitizing apparatus of claim 18 wherein said closed bottom of said cartridge base defines a plurality of apertures therethrough.

24. A sponge sanitizing apparatus comprising:
a tray defining a shallow basin sized for retaining a defined quantity of liquid and for receiving a sponge therein;
first and second receptacles integrally formed in said tray, each said receptacle defining a cavity for receiving a sterilant therein, and each said receptacle further defining a void fluidically communicative with said basin and said cavity to allow the free flow of liquid between said cavity and said basin; and
a first sanitizing cartridge with a first sterilant received in said first receptacle, said first sanitizing cartridge defining at least one aperture therethrough and in registration with said first void; and
a second sanitizing cartridge with a second sterilant of higher intensity than said first sterilant, said second cartridge received in said second receptacle, said second sanitizing cartridge defining at least one aperture therethrough and in registration with said second void.
